(19) United States
(12) Patent Application Publication
(10) Pub. No.: US 2023/0301729 A1
(43) Pub. Date: Sep. 28, 2023

(54) SURGICAL SYSTEM

(71) Applicant: MINMAXMEDICAL, Saint-Martin-D'hères (FR)

(72) Inventor: Christophe DEHAN, SAINT MARTIN D'HERES (FR)

(21) Appl. No.: 18/124,211

(22) Filed: Mar. 21, 2023

(30) Foreign Application Priority Data
Mar. 23, 2022 (EP) 22305352.1

(51) Int. Cl. A61B 34/30; A61B 34/20

(52) U.S. Cl. CPC A61B 34/30; A61B 34/20; A61B 2034/2046

(57) ABSTRACT

The present disclosure concerns a surgical system, comprising at least:
a control unit,
a storage unit configured to communicate with the control unit;
a robotic arm comprising:
   at least three motorized joints, the control unit being configured to determine, in real-time, a pose of each motorized joint of the at least three motorized joints; and
   a flange;
a passive arm attached to the flange of the robotic arm, the passive arm extending within a first plane, the passive arm comprising at least three distinct parallel axes forming a plurality of passive joints, each passive joint comprising an encoder configured to:
   determine, in real-time, an angular position of each passive joint of the plurality of passive joints; and
   send to the control unit a first corresponding information of the angular position of each passive joint of the plurality of passive joints;
a surgical device attached to the passive arm, the surgical device extending within a second plane, the second plane being parallel to the first plane, the surgical device being configured to treat a region of interest of an anatomical structure according to a predefined surgical plane, a relative pose of the surgical plane with respect to the region of interest being stored in the storage unit; and
a localization unit comprising at least:
   a first tracker attached to a segment of the robotic arm or to a base of the robotic arm; and
   a second tracker attached to the region of interest, the localization unit being configured to:
      determine, in real-time, a relative pose of the segment or the base of the robotic arm with respect to the region of interest; and
      send to the control unit a second corresponding information of the relative pose of the segment or the base of the robotic arm with respect to the region of interest,
wherein the control unit is configured to:
   determine a relative pose of the flange of the robotic arm with respect to the region of interest based on the second corresponding information of the relative pose of the segment or the base of the robotic arm with respect to the region of interest and/or on the pose of pose of each motorized joint of the at least three motorized joints, and/or on a geometry of the robotic arm being stored in the storage unit; and
   determine a relative pose of the surgical device with respect to the surgical plane, based on the relative pose of the flange of the robotic arm with respect to the region of interest, on the first corresponding information of the angular position of each passive joint of the plurality of passive joints, on the pose of the surgical plane with respect to the region of interest, and on a geometry of the passive arm stored in the storage unit and a geometry of the surgical device stored on the storage unit.

### FIELD

The present relates generally to the domain of the surgical systems.

### BACKGROUND

The use of bone attached templates and guides to guide hand-held saw or other tools, limits resulting accuracy to surgeons' dexterity, in particular due to inaccuracy in guide placement and stability over patient anatomy and mandatory free mechanical play between tool and guide. Guide placement is intrusive as it also requires large access opening and fixation over bony structure. Surgical systems to assist in cutting a plane in a bone have been proposed to provide better accuracy, lower invasiveness, and lower complication rates, all contributing to expected better patient outcomes.

In a first approach, some surgical systems may carry a cutting guide, aligned with the plane to be cut. A drawback with such surgical systems is that the cutting guide adds inaccuracy.

In a second approach, some surgical systems may carry a planar mechanism to which a surgical device is attached, the deviations of the surgical device from the planned plane are compensated thanks to a localization unit. A drawback with such surgical systems is that a tracker of the localization unit may be soiled by blood or tissue projections which adds inaccuracy or can be dangerous during such a surgical procedure.

Thus, it remains a need for an improved surgical system to maintain the precise pose of the surgical device with respect to the planned cutting plane.

### SUMMARY

An object of the present disclosure concerns a surgical system, comprising at least:
a control unit,
a storage unit configured to communicate with the control unit;
a robotic arm comprising:
   at least three motorized joints, the control unit being configured to determine, in real-time, a pose of each motorized joint of the at least three motorized joints; and
   a flange;
a passive arm attached to the flange of the robotic arm, the passive arm extending within a first plane, the passive arm comprising at least three distinct parallel axes forming a plurality of passive joints, each passive joint comprising an encoder configured to:
   determine, in real-time, an angular position of each passive joint of the plurality of passive joints; and
   send to the control unit a first corresponding information of the angular position of each passive joint of the plurality of passive joints;
a surgical device attached to the passive arm, the surgical device extending within a second plane, the second plane being parallel to the first plane, the surgical device being configured to treat a region of interest of an anatomical structure according to a predefined surgical plane, a relative pose of the surgical plane with respect to the region of interest being stored in the storage unit; and
a localization unit comprising at least:
   a first tracker attached to a segment of the robotic arm or to a base of the robotic arm; and
   a second tracker attached to the region of interest, the localization unit being configured to:
      determine, in real-time, a relative pose of the segment or the base of the robotic arm with respect to the region of interest; and
      send to the control unit a second corresponding information of the relative pose of the segment or the base of the robotic arm with respect to the region of interest,
wherein the control unit is configured to:
   determine a relative pose of the flange of the robotic arm with respect to the region of interest based on the second corresponding information of the relative pose of the segment or the base of the robotic arm with respect to the region of interest and/or on the pose of pose of each motorized joint of the at least three motorized joints, and/or on a geometry of the robotic arm being stored in the storage unit; and
   determine a relative pose of the surgical device with respect to the surgical plane, based on the relative pose of the flange of the robotic arm with respect to the region of interest, on the first corresponding information of the angular position of each passive joint of the plurality of passive joints, on the pose of the surgical plane with respect to the region of interest, and on a geometry of the passive arm stored in the storage unit and a geometry of the surgical device stored on the storage unit.

As the robotic arm comprises at least three motorized joints, it also comprises several segments connected to one another thanks to these motorized joints. Thus, each segment of the robotic arm is delimited by two adjacent motorized joints.

The control unit is configured to determine, in real-time, the poses of each motorized joint, for instance thanks to encoders housed within said motorized joint, or based on a pose of each motor driving said motorized joints. Obviously, any other known method could be implemented within the scope of the invention.

In one aspect of the present disclosure, “real-time” here means “at a predetermined frequency greater than 100 Hz”.

By “geometry” we here mean that the storage unit stores fixed and conformable dimensions and relations—such as by joint motion—between each part of the robotic arm with respect to one another, between each part of the passive arm with respect to one another between the parts of the robotic arm and of the passive arm which are connected to one another and also between part(s) of the passive arm and part(s) of the surgical device which are connected to each other. This definition applies for the whole document, adapted to the concerned objects.

The invention may be complemented by one or several of the following features, alone or in combination.

The encoders of the passive arm may be absolute encoders. According to this feature, the stored geometry of the passive arm is recorded with the corresponding values given by the encoders for each recorded geometry.

The control unit is further configured to:
determine if an active portion of the surgical device is within the surgical plane;
compute at least one instruction to be sent to at least one motorized joint of the at least three motorized joints based on the relative pose of the surgical device with respect to the surgical plane, when the active portion of the surgical device is outside the surgical plane; and
send the at least one instruction to the at least one motorized joint of the at least three motorized joints, the execution of the at least one instruction resulting in that the active portion of the surgical device is brought back within the surgical plane.

The surgical system comprises at least one display device, the control unit being configured to compute a representation, in real-time, of a pose of the region of interest and a pose of the surgical device and to send an instruction to the display device, the execution of the instruction resulting in the display of the representation. Optionally, the control unit is configured to update the representation of the pose of the region of interest as the surgical device treats the region of interest.

The display device can be mounted on a base of the surgical system from which extends the robotic arm.

The display device can be mounted on an arm attached to the robotic arm.

The display device is an augmented reality display device configured to be interposed between the region of interest and eyes of a user of the system.

The surgical device comprises a cutting tool attached to the surgical system, the cutting tool being configured to cut bony structures. Optionally, the cutting tool comprises at least one of a saw, a burr, a drill, a laser, a water jet, a scalpel, focused ultrasounds, ora shaver.

At least one passive joint of the plurality of passive joints comprises a brake housed in the at least one passive joint of the plurality of passive joints, the brake being configured to stop a motion of the at least one passive joint of the plurality of passive joints.

According to an aspect of the disclosure, the surgical system comprises at least one sensor configured to determine, in real-time, a strain and/or a force and/or a torque applied to the robotic arm and/or to the passive arm, and to send to the control unit a third corresponding information relative to a quantification of the strain and/or the force and/or the torque applied to the robotic arm and/or to the passive arm.

The control unit may be further configured to:
determine a deformation of the robotic arm and/or of the passive arm, based on the strain and/or the force and/or the torque applied to the robotic arm and/or to the passive arm determined by the at least one sensor, on a model of deformation of the robotic arm stored in the storage unit and/or on a model of the passive arm stored in the storage unit, on the first corresponding information of the angular position of each passive joint of the plurality of passive joints, on the geometry of the passive arm stored in the storage unit and on the geometry of the surgical device stored on the storage unit; and
adjust the relative pose of the surgical device with respect to the surgical plane based on the deformation of the robotic arm and/or of the passive arm.

Advantageously, the control unit may be further configured to:
determine if an active portion of the surgical device is within the surgical plane,
compute at least one instruction to be sent to at least one motorized joint of the at least three motorized joints based on the adjusted relative pose of the surgical device with respect to the surgical plane and on the deformation of the robotic arm and/or of the passive arm, when the surgical device is outside the surgical plane,
send the at least one instruction to the at least one motorized joint of the at least three motorized joints, the execution of the at least one instruction resulting in that the surgical device is brought back within the surgical plane.

The control unit can also may be further configured to:
receive the third corresponding information relative to a quantification of the strain and/or the force and/or the torque applied to the robotic arm and/or to the passive arm;
compare the third corresponding information relative to a quantification of the strain and/or the force and/or the torque applied to the robotic arm and/or to the passive arm with a first predefined threshold; and
send sensory feedback to the user when the third corresponding information relative to a quantification of the strain and/or the force and/or the torque applied to the robotic arm and/or to the passive arm exceeds the first predefined threshold.

The control unit may be further configured to:
receive the third corresponding information relative to a quantification of the strain, and/or the force and/or the torque applied to the robotic arm and/or to the passive arm;
compare the third corresponding information relative to a quantification of the strain and/or the force and/or the torque applied to the robotic arm and/or to the passive arm with a second predefined threshold; and
send an instruction to stop the surgical device when the third corresponding information relative to a quantification of the strain and/or the force and/or the torque applied to the robotic arm and/or to the passive arm exceeds the second predefined threshold.

The control unit may be further configured to:
receive the third corresponding information relative to a quantification of the strain, and/or the force and/or the torque applied to the robotic arm and/or to the passive arm;
compare the third corresponding information relative to a quantification of the strain and/or the force and/or the torque applied to the robotic arm and/or to the passive arm with a third predefined threshold; and
send an instruction to activate the brake when the third corresponding information relative to a quantification of the strain and/or the force and/or the torque applied to the robotic arm and/or to the passive arm exceeds the third predefined threshold.

It is understood that the information relative to the quantification of strains, forces and/or torques applied to the robotic arm and/or to the passive arm is provided by the at least one sensor evoked above.

According to the invention, the at least one sensor comprises at least one of:
a force sensor,
a torque sensor,
a strain gauge,
a plurality of piezoelectric or piezo-resistive gauges,
an optic fiber strain measurement device.

The at least one sensor may be arranged at one or several of the following positions:
between the flange of the robotic arm and the passive arm,
between the passive arm and the surgical device,
on at least one link of the passive arm, such link being delimited by two adjacent passive joints,

Alternatively or additionally, the at least one sensor may comprise at least two relative displacement sensors, arranged on either side of each passive joint of the plurality of passive joints of the passive arm.

### BRIEF DESCRIPTION OF THE DRAWINGS

FIG. 1 is a general perspective view of a surgical system according to an embodiment of the present disclosure.

FIG. 2 is an enlarged view of a surgical device of a surgical system treating a region of interest according to a surgical plane.

FIGS. 3a and 3b illustrate two different examples of sensors positions included to a surgical system.

### DETAILED DESCRIPTION

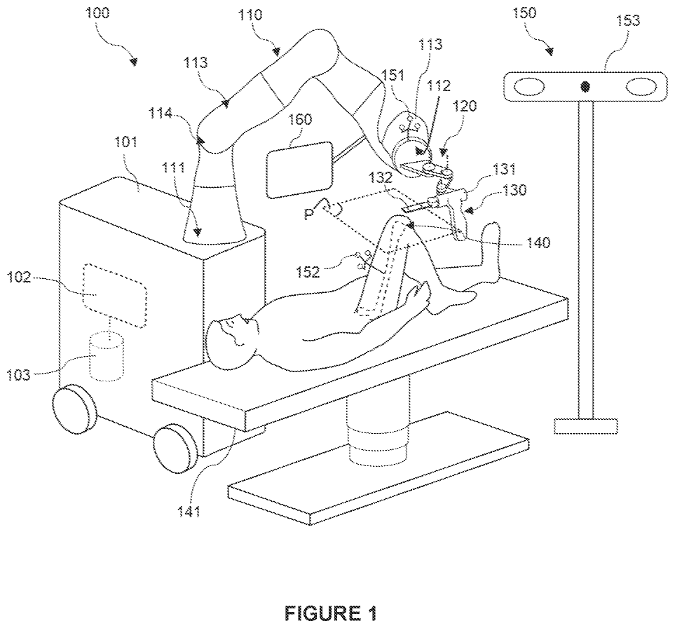

FIG. 1 is a general view of a surgical system 100 according to the disclosure. The surgical system 100 comprises a base 101 from which extends a robotic arm 110 connected to a passive arm 120. For instance, the base 101 can be a wheeled cart. As shown, a surgical device 130 is attached to the passive arm 120. As detailed below, the surgical device 130 is configured to treat a region of interest 140 according to a surgical plane P. According to the illustrated embodiment, the surgical device 130 comprises at least one power tool 131 configured to power a cutting tool 132. By “cutting tool” we here mean that the tool is configured to cut bony structures. For instance, this cutting tool can be saw, a burr, a drill, a laser, a water jet, a scalpel, focused ultrasounds or a shaver. Advantageously, the surgical device can be removably attached to the passive arm 120, so that it can be easily replaced by a linear guide configured to guide a cutting instrument.

The robotic arm 110 extends between a first end 111 connected to the base 101 and a second end 112 which forms a flange of the robotic arm 110 to which the passive arm 120 is connected. The robotic arm 110 comprises several segments 113 connected to one another thanks to motorized joints 114—only one of each being referenced on FIG. 1. According to the disclosure, the robotic arm 110 comprises at least three motorized joints 114 and it could comprise more than three motorized joints, for instance six motorized joints within the scope of the disclosure. Obviously, the robotic arm could also comprise more than six motorized joints without departing from the scope of the disclosure.

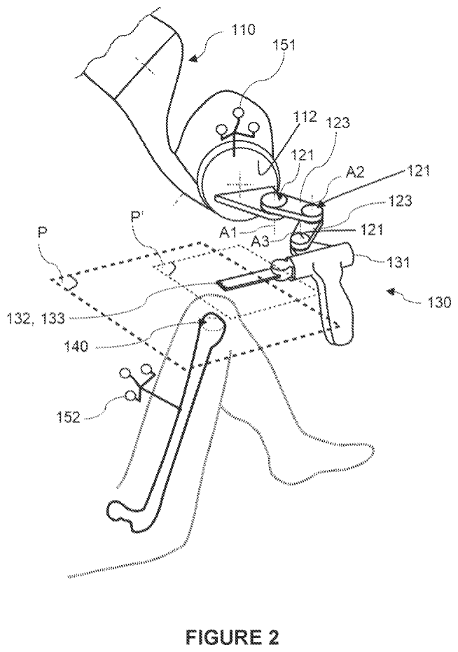

The passive arm 120 can be freely moved within a plane, hereafter referred to as the “passive arm plane”. As shown on FIG. 2, the surgical device 130 is attached to the passive arm 120 so that such surgical device 130 extends, mainly, within a plane P′ parallel to the passive arm plane. The passive arm 120 comprises at least three axes A1, A2, A3 forming three distinct passive joints 121. The words “passive joints” are here used as opposed to “motorized joints” as they are manually displaceable and deprived of motors. Two adjacent passive joints 121 define one link 123 of the passive arm 120. Consequently, the passive arm 120 comprises at least two links 123.

According to an aspect of the disclosure, the robotic arm 110 is configured to displace the passive arm 120 until the plane P′ wherein the surgical device 130 mainly extends matches the surgical plane P. Especially, at least an active portion 133 of the surgical device 130 must be within the surgical plane P. By “active portion” we here mean the portion of the surgical tool which is configured to actually cut the region of interest. According to the illustrated embodiment, the surgical device is formed as a surgical saw, the active portion being formed by a blade of such surgical saw. A user of the system can thus manipulate the surgical device 130 while ensuring that he/she is treating the region of interest 140 according to the predefined surgical plane P. As the surgical device 130 is maintained within the surgical plane P thanks to the robotic arm 110, the user of the system can operate the surgical device 130 with high precision and little effort. Thus, his/her sensitivity about the performed procedure is increased as the force feedback he/she receives is produced by the effort actually applied on the region of interest. Such a configuration is for instance illustrated on FIG. 2.

The surgical system 100 also comprises at least one control unit 102 and at least one storage unit 103, the control unit 102 being configured to communicate with the storage unit 103. This communication can be wired or wireless without departing from the scope of the disclosure. According to the illustrated embodiment, the control unit 102 and the storage unit 103 are encompassed within the base 101 of the system, but they could be positioned anywhere else within the system, or even be deported within the scope of the disclosure. The storage unit 103 is advantageously used to store one or several of the following:
geometries of the robotic arm, of the passive arm and/or of the surgical device
a relative pose of the surgical plane with respect to the region of interest
a model of deformation of the robotic arm and/or of the passive arm
a model of deformation of at least one force or torque sensor.

By “geometries” we here mean that the storage unit stores fixed and conformable dimensions and relations—such as by joint motion—between each part of the robotic arm with respect to one another, between each part of the passive arm with respect to one another between the parts of the robotic arm and of the passive arm which are connected to one another and also between part(s) of the passive arm and part(s) of the surgical device which are connected to each other. This definition applies mutatis mutandis in the whole document.

The relative pose of the surgical plane P with respect to the region of interest 140 is determined during a planning procedure. Several well-known methods can be used to realize such planning and they are not developed in this document.

As known by the skilled man of the art, the model of deformation of the robotic arm and/or of the passive arm can be determine by construction, or it can be determined later by conducting calibration tests with different loads applied at the end of the concerned arm.

Moreover, the control unit 102 is configured to determine, in real-time, a pose of each motorized joint of the robotic arm. For instance, encoders can be housed within said motorized joints, or the control unit 102 can be connected to motors driving the motorized joints, thus permitting such control unit 102 to know, at any time, the current pose of the motors and to determine, consequently, the pose of each motorized joint.

As shown, the surgical system 100 further comprises a localization unit 150. According to the illustrated embodiment, this localization unit 150 is an optical localization unit, but it could be of any other known technology, such as an electromagnetic localization unit for instance, without departing from the scope if the disclosure. The localization unit comprises at least a first tracker 151 rigidly fixed to the robotic arm 110, a second tracker 152 rigidly attached to the region of interest 140 and a camera system 153 configured to detect the current pose of the trackers 151, 152 and to determine, based on said current poses of the trackers and on a known geometry between each tracker and the corresponding tracked objects, the relative pose of the robotic arm 110 with respect to the region of interest. The localization unit is further configured to communicate with the control unit 102.

As schematically illustrated, the first tracker 151 can for instance be attached to the last segment 113 of the robotic arm 110, i.e., the segment forming the flange 112 to which the passive arm is connected. Alternatively, the first tracker 151 could be arranged on the second to last segment. According to another alternative, the first tracker 151 could be arranged on the base 101 of the surgical system 100 within the scope of the disclosure. The localization unit 150 is configured to determine, in real-time, a relative pose of the segment or the base of the robotic arm 110 to which the first tracker 151 is attached, with respect to the region of interest 140, and to send a corresponding information to the control unit 102.

The passive arm 120 further comprises at least three encoders housed within the passive joints 121. According to an aspect of the disclosure, each passive joint 121 is equipped with one of the encoders. The encoders are configured to determine, in real-time an angular position of the passive joint 121 to which it is associated, and to send a corresponding information to the control unit 102. According to an aspect of the disclosure, the encoders can be absolute encoders. If so, the storage unit 103 also stores the values of each encoder with respect to specific geometries of the passive arm. Optionally, at least one brake can be housed in at least one of the passive joints 121. For instance, this brake can be an electromagnetic brake.

Finally, the surgical system 100 comprises at least one display device 160 configured to display a representation computed by the control unit 102 of the current poses of the robotic arm 110 and of the region of interest 140. Thus, the user of the system can appreciate, at any time the relative pose of said objects. Advantageously, the representation of the region of interest can be updated, by the control unit 102, as the surgical device treats said region of interest. For instance, parts of the region of interest which have already been treated, i.e., parts where the surgical device 130 has already been operated, can be represented with different colors or textures. This is achieved thanks to an indirect tracking of the surgical device with respect to the region of interest that will be explained with more details below. According to the illustrated embodiment, the display device 160 is form as a display arranged on arm attached to the robotic arm 110. Obviously, this is merely an example and the display could be positioned on another part of the surgical system, such as its base, within the scope of the disclosure. Alternatively, the display could also be attached to an operating table 141 where a patient lies. According to yet another alternative, the display device could be an augmented reality device configured to be positioned between the eyes of the user and the region of interest, such as augmented reality glasses, goggles, or panel.

As evoked above, the surgical system of the disclosure is configured to help a user performing a planar cut on the region of interest. For instance, such system can thus be used for performing an osteotomy, or to prepare a bone to receive an artificial implant for instance in the case of a total or partial knee arthroplasty, or of a hip arthroplasty or an ankle arthroplasty etc. . . . First of all, the control unit 102 is configured to compute instruction(s) to be sent to at least one of the motorized joints, the execution of which resulting in a displacement of the robotic arm 110 so that the plane P′ wherein the surgical device extends matches the surgical plane P. A user of the system can then operate the surgical device along the surgical plane P and perform the planned cut safely.

As the user manipulates the passive arm 120, for instance by grasping the surgical device 130, he/she can apply inadvertently, forces and/or torques on the passive arm 120 which could result in a deviation of the surgical device 130, and especially of the active portion 133 of such surgical device 130, which could then be positioned outside the surgical plane P.

The localization unit 150 monitors, continuously, the relative pose of the segment 113 or the base 101 of the robotic arm 110 to which the first tracker 151 is attached, with respect to the region of interest 140 and sends corresponding information to the control unit 102. Based on this information, and/or on the pose of the motorized joints and/or on the stored geometry of the robotic arm, the control unit 102 is configured to determine a relative pose of the flange 112 of the robotic arm 110 with respect to the region of interest 140.

It is understood that the information needed to determine the relative pose of the flange 112 with respect to the region of interest 140 are different depending on the position of the first tracker 151. For instance, if the first tracker 151 is attached to the last segment 113 of the robotic arm 110, the information provided by the localization unit 150 alone is sufficient. But if the first tracker 151 is attached to the base 101 of the robotic arm 110, then the control unit 102 needs the information provided by the localization unit 150, combined with the pose of the motorized joints 114, combined with the stored geometry of the robotic arm 110 to determine the current pose of the flange 112 with respect to the region of interest 140.

Then, the control unit 102 is configured to determine the relative pose of the surgical device 130 with respect to the surgical plane P, based the determined pose of the flange 112 with respect to the region of interest 140, on the angular positions of the passive arm's joint transmitted by the encoders, on the stored relative pose of the surgical plane P with respect to the region of interest 140 and on stored geometries of the passive arm 120 and of the surgical device 130.

The system 100 thus permits to track the surgical device 130 without needing to attach a tracker on such surgical device 130. As the geometry of the surgical device 130 is stored, the control unit 102 can determine the pose of the active portion 133 of such surgical device 130 with respect to the surgical plane P based on the determined pose of said surgical device 130.

As the system is configured to ensure that the surgical device 130, and especially its active portion 133, remains within the surgical plane P, the control unit 102 is further configured to compute at least one instruction and to send it/them to at least one of the motorized joints, the execution of this instruction(s) resulting in that the active portion of the surgical device 130 is brought back within the surgical plane P.

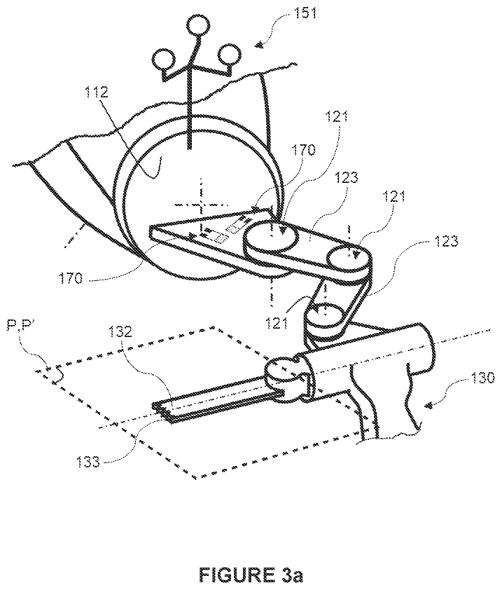

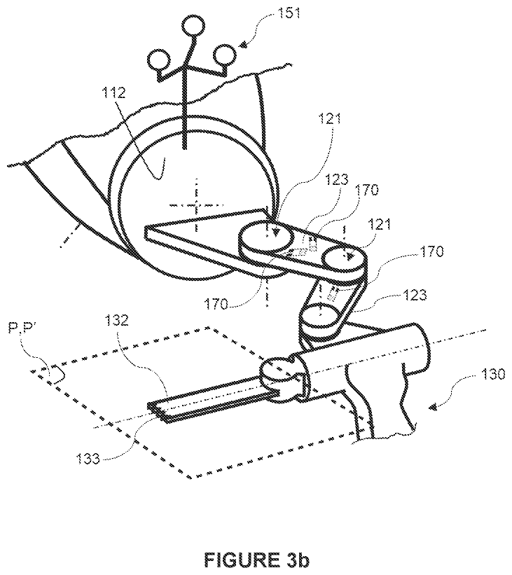

As for instance illustrated on FIGS. 3a, 3b, the surgical system 100 of the disclosure can comprise at least one sensor 170 configured to determine strains and/or torques and/or forces applied on the robotic arm 110 and/or on the passive arm 120. FIGS. 3a and 3b differs from one another in the positioning of such sensors. FIG. 3a illustrates a first example wherein the at least one sensor 170 comprises two strain gauges arranged between the flange 112 of the robotic arm 110 and the passive arm 120. FIG. 3b illustrates a second example wherein the at least one sensor 170 comprises three strain gauges arranged on both links 123 of the passive arm 120. Especially, a first strain gauge is arranged on a last link 123 of the passive arm, i.e., the link to which is connected the surgical device 130 and two other strain gauges are arranged on a first link 123 of the passive arm, i.e., the link connected to the robotic arm 110.

It is understood that those strain gauges could be replaced by any other known strains, torques or forces sensor within the scope of the disclosure. Examples of strains, forces or torques sensors compatible with the present disclosure are given below. Also, the surgical system could comprise more or less sensors than illustrated without departing from the scope of the disclosure.

Obviously, those are only examples of how to carry the disclosure and the at least one sensor could be in another position with respect to the system within the scope of the disclosure. Also, the system could comprise several sensors or different kind at several locations without departing from the scope of the disclosure.

When the system is equipped with such sensor(s) 170, the control unit 102 is further configured to determine a deformation of the robotic arm 110 and/or of the passive arm 120, based on the determined strains and/or forces and/or torques applied to the robotic arm and/or to the passive arm, on the angular positions of the passive joints transmitted by the encoders and on the stored model of deformation of the robotic arm and/or of the passive arm. The control unit 102 is then configured to adjust the previously determined relative pose of the surgical device 130 with respect to the surgical plane P based on said determined deformation.

In one embodiment, adjusting the previously determined relative pose of the surgical device 130 with respect to the surgical plane P based on said determined deformation corresponds to compute a current relative pose of the surgical device 130 with respect to the surgical plane P as a function of the previously determined relative pose of the surgical device 130 with respect to the surgical plane P and of the determined deformation.

The control unit 102 can be further configured to determine whether the surgical device 130, and especially its active portion 133, is within the surgical plane P or not. When the active portion 133 of the surgical device 130 is outside the surgical plane P, the control unit 102 is configured to compute at least one instruction to be sent to at least one of the motorized joints in order to bring back the active portion 133 of the surgical device 130 within the surgical plane P. This at least one instruction is computed based on the adjusted relative pose of the surgical device 130 with respect to the surgical plane P and on the computed deformation of the robotic arm and/or of the passive arm. The instruction(s) can then be sent to at least one of the motorized joints in order to bring the active portion 133 of the surgical device 130 back within the surgical plane P.

The control unit 102 can also be configured to compare the strains and/or forces and/or torques applied to the robotic arm and to the passive arm with a predefined first threshold and to provide feedback to the user when said strains, forces and/or torques exceed said first threshold. This feedback can for instance be tactile feedback, visual feedback or auditive feedback. Obviously, any other sensorial feedback could be used without departing from the scope if the disclosure.

Additionally, the control unit 102 can be configured to compare the strains, forces and/or torques applied to the robotic arm with a second threshold, greater than the first threshold, and to stop the surgical device to prevent any unwanted cut when the strains, forces and/or torques are above this second threshold. Additionally or alternatively, the control unit can be configured to activate the brakes of the passive joints when the strains, forces and/or torques are above a third threshold, this third threshold being greater than the first threshold and than the second threshold.

According to different embodiments of the disclosure, the at least one sensor 170 can comprise one or several of the following:
a force sensor,
a torque sensor,
strain gauge,
piezoelectric or piezo-resistive gauges,
optic fiber strain measurement device.

According to the disclosure, the at least one sensor 170 can be arranged at one or several of the following positions:
between the flange 112 of the robotic arm 110 and the passive arm 120, as illustrated on FIG. 3a,
between the passive arm 120 and the surgical device 130,
on at least one link 123 of the passive arm 120, as illustrated on FIG. 3b.

According to an aspect of the disclosure, the at least one sensor 170 can additionally or alternatively comprise at least two relative displacement sensors, arranged on either side of each passive joint 121 of the passive arm 120. By monitoring the relative displacement of the links 123 connected by the concerned passive joint 121, the relative displacement sensors permit to detect deflection and torsion applied on either of said links.

Obviously, the surgical system 100 could comprise a combination of those without departing from the scope of the disclosure.

1. A surgical system, comprising at least:
a control unit,
a storage unit configured to communicate with the control unit;
a robotic arm comprising:
at least three motorized joints, the control unit being configured to determine, in real-time, a pose of each motorized joint of the at least three motorized joints; and
a flange;
a passive arm attached to the flange of the robotic arm, the passive arm extending within a first plane, the passive arm comprising at least three distinct parallel axes forming a plurality of passive joints, each passive joint comprising an encoder configured to:
determine, in real-time, an angular position of each passive joint of the plurality of passive joints; and
send to the control unit a first corresponding information of the angular position of each passive joint of the plurality of passive joints;
a surgical device attached to the passive arm, the surgical device extending within a second plane, the second plane being parallel to the first plane, the surgical device being configured to treat a region of interest of an anatomical structure according to a predefined surgical plane, a relative pose of the surgical plane with respect to the region of interest being stored in the storage unit; and
a localization unit comprising at least:
a first tracker attached to a segment of the robotic arm or to a base of the robotic arm; and
a second tracker attached to the region of interest, the localization unit being configured to:
determine, in real-time, a relative pose of the segment or the base of the robotic arm with respect to the region of interest; and
send to the control unit a second corresponding information of the relative pose of the segment or the base of the robotic arm with respect to the region of interest,
wherein the control unit is configured to:
determine a relative pose of the flange of the robotic arm with respect to the region of interest based on the second corresponding information of the relative pose of the segment or the base of the robotic arm with respect to the region of interest and/or on the pose of pose of each motorized joint of the at least three motorized joints, and/or on a geometry of the robotic arm being stored in the storage unit; and
determine a relative pose of the surgical device with respect to the surgical plane, based on the relative pose of the flange of the robotic arm with respect to the region of interest, on the first corresponding information of the angular position of each passive joint of the plurality of passive joints, on the pose of the surgical plane with respect to the region of interest, and on a geometry of the passive arm stored in the storage unit and a geometry of the surgical device stored on the storage unit.

2. The surgical system of claim 1, wherein the control unit is further configured to:
determine if an active portion of the surgical device is within the surgical plane;
compute at least one instruction to be sent to at least one motorized joint of the at least three motorized joints based on the relative pose of the surgical device with respect to the surgical plane, when the active portion of the surgical device is outside the surgical plane; and
send the at least one instruction to the at least one motorized joint of the at least three motorized joints, the execution of the at least one instruction resulting in that the active portion of the surgical device is brought back within the surgical plane.

3. The surgical system of claim 1, comprising at least one display device, the control unit being configured to compute a representation, in real-time, of a pose of the region of interest and a pose of the surgical device and to send an instruction to the display device, the execution of the instruction resulting in the display of the representation.

4. The surgical system of claim 3, wherein the control unit is configured to update the representation of the pose of the region of interest as the surgical device treats the region of interest.

5. The surgical system of claim 3, wherein the display device is an augmented reality display device configured to be interposed between the region of interest and eyes of a user of the system.

6. The surgical system of claim 1, wherein the surgical device comprises a cutting tool attached to the surgical system, the cutting tool being configured to cut bony structures.

7. The surgical system of claim 1, wherein the cutting tool comprises at least one of a saw, a burr, a drill, a laser, a water jet, a scalpel, focused ultrasounds, or a shaver.

8. The surgical system of claim 1, wherein the surgical device is removable from the passive arm and can be replaced by a linear guide configured to be attached to the passive arm, the linear guide being configured to guide a cutting tool.

9. The surgical system of claim 1, wherein at least one passive joint of the plurality of passive joints comprises a brake housed in the at least one passive joint of the plurality of passive joints, the brake being configured to stop a motion of the at least one passive joint of the plurality of passive joints.

10. The surgical system of claim 1, further comprising at least one sensor configured to determine, in real-time, a strain and/or a force and/or a torque applied to the robotic arm and/or to the passive arm, and to send to the control unit a third corresponding information relative to a quantification of the strain and/or the force and/or the torque applied to the robotic arm and/or to the passive arm.

11. The surgical system of claim 10, wherein the control unit is further configured to:
determine a deformation of the robotic arm and/or of the passive arm, based on the strain and/or the force and/or the torque applied to the robotic arm and/or to the passive arm determined by the at least one sensor, on a model of deformation of the robotic arm stored in the storage unit and/or on a model of the passive arm stored in the storage unit, on the first corresponding information of the angular position of each passive joint of the plurality of passive joints, on the geometry of the passive arm stored in the storage unit and on the geometry of the surgical device stored on the storage unit; and
adjust the relative pose of the surgical device with respect to the surgical plane based on the deformation of the robotic arm and/or of the passive arm.

12. The surgical system of claim 11, wherein the control unit is further configured to:
determine if an active portion of the surgical device is within the surgical plane,
compute at least one instruction to be sent to at least one motorized joint of the at least three motorized joints based on the adjusted relative pose of the surgical device with respect to the surgical plane and on the deformation of the robotic arm and/or of the passive arm, when the surgical device is outside the surgical plane,
send the at least one instruction to the at least one motorized joint of the at least three motorized joints, the execution of the at least one instruction resulting in that the surgical device is brought back within the surgical plane.

13. The surgical system of claim 10, wherein the control unit is further configured to:
receive the third corresponding information relative to a quantification of the strain and/or the force and/or the torque applied to the robotic arm and/or to the passive arm;
compare the third corresponding information relative to a quantification of the strain and/or the force and/or the torque applied to the robotic arm and/or to the passive arm with a first predefined threshold; and
send sensory feedback to the user when the third corresponding information relative to a quantification of the strain and/or the force and/or the torque applied to the robotic arm and/or to the passive arm exceeds the first predefined threshold.

14. The surgical system of claim 10, wherein the control unit is further configured to:
receive the third corresponding information relative to a quantification of the strain, and/or the force and/or the torque applied to the robotic arm and/or to the passive arm;
compare the third corresponding information relative to a quantification of the strain and/or the force and/or the torque applied to the robotic arm and/or to the passive arm with a second predefined threshold; and
send an instruction to stop the surgical device when the third corresponding information relative to a quantification of the strain and/or the force and/or the torque applied to the robotic arm and/or to the passive arm exceeds the second predefined threshold.

15. The surgical system of claim 10, wherein at least one passive joint of the plurality of passive joints comprises a brake housed in the at least one passive joint of the plurality of passive joints, the brake being configured to stop a motion of the at least one passive joint of the plurality of passive joints, wherein the control unit is further configured to:
receive the third corresponding information relative to a quantification of the strain, and/or the force and/or the torque applied to the robotic arm and/or to the passive arm;
compare the third corresponding information relative to a quantification of the strain and/or the force and/or the torque applied to the robotic arm and/or to the passive arm with a third predefined threshold; and
send an instruction to activate the brake when the third corresponding information relative to a quantification of the strain and/or the force and/or the torque applied to the robotic arm and/or to the passive arm exceeds the third predefined threshold.

16. The surgical system of claim 10, wherein the at least one sensor comprises at least one of:
a force sensor,
a torque sensor,
a strain gauge,
a plurality of piezoelectric or piezo-resistive gauges,
an optic fiber strain measurement device.

17. The surgical system of claim 10, wherein the at least one sensor is arranged between the flange of the robotic arm and the passive arm.

18. The surgical system of claim 10, wherein the at least one sensor is arranged between the passive arm and the surgical device.

19. The surgical system of claim 10, wherein the at least one sensor is arranged on at least one link of the passive arm, such link being delimited by two adjacent passive joints.

20. The surgical system of claim 10, wherein the at least one sensor comprises at least two relative displacement sensors, arranged on either side of each passive joint of the plurality of passive joints of the passive arm.
